(19) United States
(12) Patent Application Publication
(10) Pub. No.: US 2012/0004119 A1
(43) Pub. Date: Jan. 5, 2012

(54) GENE EXPRESSION MARKERS OF ONCOLYTIC VIRUS SENSITIVITY

(76) Inventors: Marc Lenburg, Brookline, MA (US); Lynda Chin, Houston, TX (US)

(21) Appl. No.: 13/172,577

(22) Filed: Jun. 29, 2011

Related U.S. Application Data
(60) Provisional application No. 61/360,119, filed on Jun. 30, 2010.

(51) Int. Cl. C40B 30/00; C40B 40/06; G01N 33/53; G01N 33/50; C12Q 1/68

(52) U.S. Cl. 506/7; 436/94; 435/6.1; 435/6.12; 436/501; 435/7.92; 506/16

(57) ABSTRACT

A method of predicting an efficacy of an oncolytic virus treatment for a tumor comprises calculating a single-gene predictor score for each of a plurality of genes; calculating a predictor score from the sum of the single-gene predictor scores for the plurality of genes; and predicting, if the predictor score is greater than a predictor score threshold, that the treatment would have efficacy, and if the predictor score is less than a predictor score threshold, that the treatment would lack efficacy. Also, methods of predicting an efficacy of a treatment for a tumor comprise identifying the type and subtype of the tumor, wherein the efficacy of the treatment for the type and subtype of the tumor is known.

Under 35 U.S.C. §119(e)(1), this application claims the benefit of U.S. provisional patent application 61/360,119, filed Jun. 30, 2010.

### BACKGROUND OF THE INVENTION

The present invention relates to cancer treatments.

### SUMMARY OF THE INVENTION

In one embodiment, the present invention relates to a method, comprising: determining an expression level for each of the plurality of genes in a tumor sample obtained from a patient; calculating a single-gene predictor score for each of the plurality of genes; calculating a total predictor score from the sum of the single-gene predictor scores for the plurality of genes; and predicting, if the total predictor score satisfies a first predictor score threshold, that an oncolytic virus treatment for the tumor would have efficacy, and if the total predictor score satisfies a second predictor score threshold, that the oncolytic virus treatment for the tumor would lack efficacy.

In one embodiment, the present invention relates to a method, comprising: identifying whether a central nervous system tumor is of a proneural subtype, a mesenchymal subtype, or a third subtype; predicting an oncolytic virus treatment to have efficacy if the tumor is of the proneural subtype; and predicting the oncolytic virus treatment to lack efficacy if the tumor is of the mesenchymal subtype.

In another embodiment, the present invention relates to a method, comprising identifying whether a lung tumor is of a small-cell subtype, a non-small-cell subtype, or a third subtype; predicting an oncolytic virus treatment to have efficacy if the tumor is of the small-cell subtype; and predicting the oncolytic virus treatment to lack efficacy if the tumor is of the non-small-cell subtype.

In one embodiment, the present invention relates to a kit for predicting an efficacy of an oncolytic virus treatment for a tumor, the kit comprising reagents for measuring an expression level for each of a plurality of genes in a tumor sample obtained from a patient, wherein the plurality of genes comprises at least two of ABI3BP, AGXT2L2, AKR1C1, ANKRD5, ANLN, ANO10, ANTXR2, ANXA1, ARC, ARL2, ATF7IP, ATP6V0A2, ATRIP, AUTS2, AXGT2L2, BAD, BCL9L, BLM, BR13, C10orf11, C14orf106, C14orf45, C15orf5, C17orf76, C1orf115, C1orf31, C20orf134, C20orf88, C21orf91, C2orf88, C4orf32, C6orf226, C7orf31, C7org106, CA14, CALCOCO2, CBX5, CBX6, CCDC109B, CCDC30, CCDC45, CD44, CD83, CDK5R1, CEP152, CHD1, CHKA, CPT1C, CPVL, CTSA, CYP4V2, DAPK1, DCDC2, DDX60L, DEPDC7, DHX33, DNM1L, DUS1L, EHD2, ETV2, F2R, F8, FAM129B, FAM174B, FAM178A, FIBP, FKBP10, FLJ10213, FSCN1, FSTL5, FTO, GALNT3, GARNL4, GMPR, GNA12, GPR137B, GPR143, GPX4, GPX7, hCG 20036, HHAT, HIST1H2BH, HK1, HMGA2, HOOK1, HOXA10, HOXA13, HOXA3, HOXA5, HOXA6, HOXB7, HOXB9, HOXB9K, HPS4, ICAM3, IGFBP3, IKAA1324L, IL12RB2, INTS2, ITGA5, ITGA7, ITM2C, ITPKB, IVNS1ABP, KCNA4, KCNK1, KCNN4, KCTD17, KIAA0355, KIAA0652, KIAA1324, KIAA1598, KIF23, LINS1, LOC152217, LOC202781, LOC401068, LPPR2, LRRC29, LVN, MAGEH1, MAN1B1, MAP4K3, MAPKAPK3, MBL1P1, MECOM, MED16, MEPCE, MESDC1, MFAP2, MGAT4A, MICAL3, MOSC2, MOSPD1, MRS2, MVP, MYADM, MYOM2, NCALD, NCOA6, NDRG1, NDUFV1, NEIL2, NELL1, NEO1, NFASC, NFIC, NINL, NME5, NNMT, NOL3, NR1D1, NRP1, NUDT22, OSCP1, OTUB1, PAEP, PCDH18, PCDH96, PCDHB11, PCDHB16, PCDHB2, PCDHB6, PCDHB8, PCF11, PDCDG2, PDCHB11, PDE8A, PFTK1, PGBD5, PGLYRP2, PHLPP2, PHPT1, PHYH, PITPNM1, PLAG1, PLCL2, PNLPA6, POGK, POLD4, POLDIP2, PPAP2C, PPM1H, PPP2R1A, PRRX1, PTMS, PTRF, PUM2, PUS3, RAB11FIP4, RAB15, RAB17, RAB1B, RABG1G, RASSF8, RBMS3, RC3H1, RCOR3, RGS10, RGS20, RNF187, ROM1, RRAS, RSPO3, RTN4, SB3BGRL3, SEMA6A, SEPT3, SERGEF, SERPINH1, SFRS15, SH3BGRL3, SH3YL1, SKA2, SLC16A6, SLC17A5, SLC27A3, SLC2A3P1, SLC44A3, SMOX, SNAPC2, SNX21, SORBS1, SOX8, SP4, SPAG4, SPAG9, ST6GALNAC, SUMF2, SYCP2, TBRG1, THBS3, THYN1, TIK2, TK1, TK2, TM4SF18, TMEM170B, TMEM60, TNFRSF1A, TNKS1BP1, TOB1, TOM1, TRPC3, TRPS1, TSC22D3, TTYH2, TUG1, UBASH3B, UBXN6, WNT5A, WT1, ZBTB41, ZBTB47, ZEB1, ZFP36L1, ZMYM2, ZNF138, ZNF175, ZNF195, ZNF280C, ZNF462, ZNF561, or ZXDB.

### BRIEF DESCRIPTION OF THE DRAWINGS

The invention may be understood by reference to the following description taken in conjunction with the accompanying drawings, in which:

FIG. 1 shows predictor scores vs. log₁₀ of the NDV IC₈₀ for a plurality of melanoma and CNS lines using a melanoma signature, a CNS signature, and a joint signature, as described in the examples.

FIG. 2 shows predictions of NDV sensitivity or resistance for lung tumor lines from the publicly available GSK data set.

FIG. 3 shows prediction of NDV sensitivity or resistance for glioblastoma tumor lines from the publicly available GSK data set.

FIG. 4 shows prediction of NDV sensitivity or resistance for primary melanoma lines and melanoma metastases.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DESCRIPTION OF ILLUSTRATIVE EMBODIMENTS

In one embodiment, the present invention relates to a method, comprising:

determining an expression level for each of the plurality of genes in a tumor sample obtained from a patient;

calculating a single-gene predictor score for each of the plurality of genes;

calculating a total predictor score from the sum of the single-gene predictor scores for the plurality of genes; and

predicting, if the total predictor score satisfies a first predictor score threshold, that an oncolytic virus treatment for the tumor would have efficacy, and if the total predictor score satisfies a second predictor score threshold, that the oncolytic virus treatment for the tumor would lack efficacy.

The word “tumor” is used herein to any neoplastic cells, not necessarily those found in a solid neoplasm, except in particular passages where the phrase or sentence including the word would indicate to the person of ordinary skill in the art that the word refers to solid neoplasms.

An oncolytic virus, as used herein, is a virus that is able to infect and lyse cancer cells. Replication of an oncolytic virus can both facilitate tumor cell destruction and produce dose amplification at the tumor.

Known oncolytic viruses include, but are not limited to, Herpes virus, Reovirus, E1B deleted adenovirus, Vesicular Stomatitis Virus, and Pox viruses. These oncolytic viruses have the potential to not only destroy tumor cells, but also release antigens from the destroyed tumor cells, thereby triggering an immune response.

In one embodiment, the treatment comprises administration of the at least one oncolytic virus. For example, the oncolytic virus may be selected from the group consisting of paramyxovirus, reovirus, herpesvirus, adenovirus, and Semliki Forest virus. In one further embodiment, the treatment comprises administration of Newcastle Disease Virus (NDV). In one yet further embodiment, the NDV is from a strain selected from the group consisting of MTH68/H, PV-701, and 73-T. Oncolytic viruses, including specific ones enumerated above, will be described in more detail below.

NDV is a negative-sense single-stranded RNA virus that causes a highly contagious disease affecting several domesticated and wild avian species. Specifically, NDV is classified as an avian paramyxovirus-1 (APMV-1) in the rubulavirus genus of the family paramyxoviridae in the order mononegaviralis. NDV is an enveloped virus of 100-300 nm diameters with a negative-sense single stranded RNA genome of roughly 16,000 nucleotides. The NDV genome contains 6 genes encoding 6 major polypeptides: L, HN, F, M, P and NP. The RNA dependent RNA polymerase involves the proteins L, P and NP which are translated in infected cells at free ribosomes in the cytoplasm. The F glycoprotein is synthesized as an inactive precursor (F0, 67 kDa), which undergoes proteolytic cleavage to yield the biologically active protein consisting of the disulfide-linked chains F1 (55 kDa) and F2 (12.5 kDa).

Various field-isolated forms of NDV have been classified according to their virulence: velogenic (highly pathogenic), mesogenic (intermediately pathogenic), or lentogenic (apathogenic).

Although NDV is generally thought to pose no hazard to human health, human exposure to the virus may lead to mild conjunctivitis and flu-like symptoms (e.g., mild fever for about 24 hr). Enveloped viruses such as NDV enter the cell through two main pathways: 1) direct fusion between the envelope and the plasma membrane and 2) receptor mediated endocytosis. For NDV, it has been established that the membrane fusion process takes place at the host plasma membrane in a pH-independent manner. Activation of the fusion protein F occurs through interaction of the viral glycoproteins with the sialic acid containing cellular receptors such as gangliosides and N-glycoproteins. In addition, it was recently shown that NDV may infect cells also through a caveolae-dependent endocytic pathway as an alternative route. A certain percentage of virions might become endocytosed to endosomes where fusion would occur at lowered pH. The ordered assembly and release of infectious NDV particles has been shown to depend on membrane lipid rafts. These are defined as cholesterol- and sphingolipid-rich microdomains in the exoplasmic leaflet of cellular plasma membranes. Newly produced virions showed an accumulation of HN, F and NP viral proteins in lipid rafts early after synthesis and contained the lipid raft-associated proteins caveolin-1, flotillin-2 and actin but not the non-lipid raft associated transferring receptor.

Various NDV strains have been found to be either lytic or nonlytic for human cells. Lytic strains generally produce activated hemagglutinin-neuraminidase and fusion protein molecules in the outer coat of progeny viruses in human cells, whereas nonlytic strains generally produce inactive versions of these molecules. Though not to be bound by theory, entry of NDV into a human cell may depend on activated hemagglutinin-neuraminidase and fusion protein molecules on the viral surface binding to sialic acid-containing molecules on the surface of a human cell.

Lytic and nonlytic strains of NDV also differ in the mechanisms by which they kill infected cells. Among lytic strains, the budding of progeny viruses containing activated hemagglutinin-neuraminidase and fusion protein molecules in their outer coats generally causes the plasma membrane of NDV-infected cells to fuse with the plasma membrane of adjacent cells, leading to the production of large, inviable fused cells (syncytia). Nonlytic strains kill infected cells more slowly, generally by viral disruption of normal host cell metabolism. The progeny virus particles made by non-lytic strains contain inactive versions of F molecules.

NDV exerts oncolysis by both intrinsic and extrinsic caspase-dependent pathways of cell death. Oncolytic NDV strains are cytotoxic to human tumor cell lines of ecto-, endo, and mesodermal origin. Such cytotoxicity is primarily due to multiple caspase dependent pathways of apoptosis. NDV triggers apoptosis by activating the mitochondrial/intrinsic pathway of apoptosis. NDV infection results in a loss of mitochondrial membrane potential and a release of mitochondrial protein cytochrome C. In addition, NDV infection leads to early activation of caspase 9 and caspase 3. In contrast, cleavage of caspase 8, which is predominantly activated by the death receptor pathway, is a TRAIL induced late event in NDV mediated apoptosis of tumor cells. The death signals generated by NDV in tumor cells ultimately converge at the mitochondria.

Either type of NDV strain generally replicates much more readily in human cancer cells than in most human cells. Certain NDV strains can replicate up to 10,000 times better in human neoplastically transformed cells than in most human cells. For example, while in non-tumorigenic human peripheral blood mononuclear cells (PBMC) the replication cycle of NDV is stopped after production of positive strand RNA, PBMC tumor cells continue in the replication cycle and copy viral genomes within 10-50 hours after infection.

Exemplary NDV strains include 73-T, MTH-68, Ulster, and NDV-HUJ. Sinkovics, et al., J. Clin. Virol. 16:1-15 (2000); Freeman, et al., Molec. Therapy 13:221 (2006); U.S. Pat. No. 7,223,389; WO 2005/051330; WO 2005/051433; Csatary, et al., Anticancer Res. 19:635-638 (1999); Csatary, et al., J. Am. Med. Assoc. 281:1588-1589 (1999); Csatary, et al., J. Neurooncol. 67:83-93 (2004).

The total predictor score can be calculated in any appropriate manner, such as principal components analysis, support vector machines, or other techniques known to the person of ordinary skill in the art having the benefit of the present disclosure. In one embodiment, the total predictor score is calculated from a plurality of single-gene predictor scores. Generally, each single-gene predictor score is calculated in view of reference expression levels of the gene in a first plurality of tumor lines or tumor tissues known to be sensitive to the oncolytic virus treatment and a second plurality of tumor lines or tumor tissues tumor lines or tumor tissues known to be resistant to the oncolytic virus treatment. In a particular embodiment of the method, each single-gene predictor score is calculated from a multiplier for the gene times (the expression level in the tumor sample minus a threshold value for the gene) divided by a weight for the gene,

Wherein:

the threshold value for each gene is equal to a measure of central tendency between reference expression levels of the gene in a first plurality of tumor lines or tumor tissues known to be sensitive to the oncolytic virus treatment and a second plurality of tumor lines or tumor tissues known to be resistant to the oncolytic virus treatment;

the weight for each gene is equal to a measure of the variability of reference expression levels within the first plurality of tumor lines or tumor tissues and within the second plurality of tumor lines or tumor tissues; and

the multiplier for each gene is equal to 1, if a measure of central tendency of the reference expression levels of the gene in the first plurality of tumor lines or tumor tissues is greater than the threshold value, or −1, if a measure of central tendency of the reference expression levels of the gene in the second plurality of tumor lines or tumor tissues is greater than the threshold value.

As stated above, it is determined whether the total predictor score satisfies a first predictor score threshold or a second predictor score threshold. The predictor score thresholds can be one or more separate values or the same value, and “satisfies” can mean is greater than, is less than, is equal to, is closer in value to a first subthreshold than to a second or vice versa, etc. In one embodiment, particularly useful in the embodiment referred to above in which each single-gene predictor score is calculated from a multiplier for the gene times (the expression level in the tumor sample minus a threshold value for the gene) divided by a weight for the gene, the total predictor score is the sum of the single-gene predictor scores, the first predictor score threshold is satisfied if the total predictor score is greater than zero, and the second predictor score threshold is satisfied if the total predictor score is less than zero.

In another embodiment, the first predictor score threshold is satisfied if the total predictor score is greater than a positive first value, and the second predictor score threshold is satisfied if the total predictor score is less than a negative second value. In this embodiment, total predictor scores less than the positive first value and greater than the negative second value can be considered to yield a prediction of intermediate sensitivity, or a finding that no prediction can be made.

It should be borne in mind that sensitivity and resistance of tumors to treatments are generally found on a continuum, with sensitive tumors generally requiring lower doses, less frequent doses, or both of an anticancer agent, such as an oncolytic virus, to yield a desired reduction in tumor mass, reduction in the growth rate of the tumor, regression of the tumor, lack of progression of the tumor, lack of or later metastasis of the tumor, or the like. If desired, the first plurality and second plurality of tumor lines or tumor tissues can be chosen from a larger pool of tumor lines or tumor tissues that includes lines or tissues from the center of the continuum that are excluded from further consideration. Also, if desired, the sensitivity or resistance of tumor lines or tumor tissues from the center of the continuum can be assayed as a validation of the correlation between expression and sensitivity/resistance.

The plurality of genes may comprise any genes and any number thereof. Generally, it is desirable for the genes to be genes which exhibit differences in expression levels between tumor lines or tumor tissues known to be sensitive to the treatment and tumor lines or tumor tissues known to be resistant to the treatment. For example, a first gene may be more highly expressed in tumor lines or tumor tissues known to be sensitive to the treatment than in tumor lines or tumor tissues known to be resistant to the treatment. For another example, a second gene may be more highly expressed in tumor lines or tumor tissues known to be resistant to the treatment than in tumor lines or tumor tissues known to be sensitive to the treatment.

The plurality of genes may be chosen from a larger pool of genes, some of which may lack significant differences in expression levels between tumor lines or tumor tissues known to be sensitive to the treatment and tumor lines or tumor tissues known to be resistant to the treatment. In one embodiment, the plurality of genes is determined by screening a panel of expression levels of a pool of genes from a number of tumor lines or tumor tissues including both sensitive and resistant lines and selecting those genes having the greatest differences in expression levels for the plurality of genes.

In one embodiment, the plurality of genes can be selected from a larger pool of genes by assuming a correlation of gene expression among tumor lines or tumor tissues that differ slightly. For example, the plurality of genes can be selected, in whole or in part, from those genes from a larger pool that are most overexpressed or underexpressed in both the three most sensitive tumor lines or tumor tissues and the four most sensitive tumor lines or tumor tissues. Genes overexpressed in the set of three most sensitive tumor lines or tumor tissues but not overexpressed in the set of four most sensitive tumor lines or tumor tissues can be considered to be false positives reflecting noisy data that gives a spurious indication of overexpression in one set. Generally, overexpression or underexpression differences for a single gene between two substantially overlapping sets of tumor lines or tumor tissues (n and n+1 most sensitive, m and m+1 ranked by IC₅₀ and IC₈₀ values, etc.) can be considered a sign of noisy data that may best be excluded from further analysis.

Generally speaking, the genes and the tumor lines or tumor tissues are of the same species as the patient, and both the first plurality of tumor lines or tumor tissues and the second plurality of tumor lines or tumor tissues are of the same type as the patient's tumor. Exemplary tumor types include, but are not limited to, tumors of the central nervous system, skin, bladder, breast, esophagus, kidney, liver, lung, ovary, pancreas, stomach, uterus, cervix, testicle, or colon, or metastases thereof.

The person of ordinary skill in the art will understand the specific genes making up the plurality of genes may be different between different tumor types, but the specific genes can be readily identified as a matter of routine experimentation in light of the teachings of the present specification.

In one embodiment, the plurality of genes comprises at least two of:

ABI3BP, AGXT2L2, AKR1C1, ANKRD5, ANLN, ANO10, ANTXR2, ANXA1, ARC, ARL2, ATF7IP, ATP6V0A2, ATRIP, AUTS2, AXGT2L2, BAD, BCL9L, BLM, BR13, C10orf11, C14orf106, C14orf45, C15orf5, C17orf76, C1orf115, C1orf31, C20orf134, C20orf88, C21orf91, C2orf88, C4orf32, C6orf226, C7orf31, C7org106, CA14, CALCOCO2, CBX5, CBX6, CCDC109B, CCDC30, CCDC45, CD44, CD83, CDK5R1, CEP152, CHD1, CHKA, CPT1C, CPVL, CTSA, CYP4V2, DAPK1, DCDC2, DDX60L, DEPDC7, DHX33, DNM1L, DUS1L, EHD2, ETV2, F2R, F8, FAM129B, FAM174B, FAM178A, FIBP, FKBP10, FLJ10213, FSCN1, FSTL5, FTO, GALNT3, GARNL4, GMPR, GNA12, GPR137B, GPR143, GPX4, GPX7, hCG 20036, HHAT, HIST1H2BH, HK1, HMGA2, HOOK1, HOXA10, HOXA13, HOXA3, HOXA5, HOXA6, HOXB7, HOXB9, HOXB9K, HPS4, ICAM3, IGFBP3, IKAA1324L, IL12RB2, INTS2, ITGA5, ITGA7, ITM2C, ITPKB, IVNS1ABP, KCNA4, KCNK1, KCNN4, KCTD17, KIAA0355, KIAA0652, KIAA1324, KIAA1598, KIF23, LINS1, LOC152217, LOC202781, LOC401068, LPPR2, LRRC29, LVN, MAGEH1, MAN1B1, MAP4K3, MAPKAPK3, MBL1P1, MECOM, MED16, MEPCE, MESDC1, MFAP2, MGAT4A, MICAL3, MOSC2, MOSPD1, MRS2, MVP, MYADM, MYOM2, NCALD, NCOA6, NDRG1, NDUFV1, NEIL2, NELL1, NEO1, NFASC, NFIC, NINL, NME5, NNMT, NOL3, NR1D1, NRP1, NUDT22, OSCP1, OTUB1, PAEP, PCDH18, PCDH96, PCDHB11, PCDHB16, PCDHB2, PCDHB6, PCDHB8, PCF11, PDCD2, PDCHB11, PDE8A, PFTK1, PGBD5, PGLYRP2, PHLPP2, PHPT1, PHYH, PITPNM1, PLAG1, PLCL2, PNLPA6, POGK, POLD4, POLDIP2, PPAP2C, PPM1H, PPP2R1A, PRRX1, PTMS, PTRF, PUM2, PUS3, RAB11FIP4, RAB15, RAB17, RAB1B, RABG1G, RASSF8, RBMS3, RC3H1, RCOR3, RGS10, RGS20, RNF187, ROM1, RRAS, RSPO3, RTN4, SB3BGRL3, SEMA6A, SEPT3, SERGEF, SERPINH1, SFRS15, SH3BGRL3, SH3YL1, SKA2, SLC16A6, SLC17A5, SLC27A3, SLC2A3P1, SLC44A3, SMOX, SNAPC2, SNX21, SORBS1, SOX8, SP4, SPAG4, SPAG9, ST6GALNAC, SUMF2, SYCP2, TBRG1, THBS3, THYN1, TIK2, TK1, TK2, TM4SF18, TMEM170B, TMEM60, TNFRSF1A, TNKS1BP1, TOB1, TOM1, TRPC3, TRPS1, TSC22D3, TTYH2, TUG1, UBASH3B, UBXN6, WNT5A, WT1, ZBTB41, ZBTB47, ZEB1, ZFP36L1, ZMYM2, ZNF138, ZNF175, ZNF195, ZNF280C, ZNF462, ZNF561, or ZXDB.

In a further embodiment, the plurality of genes comprises at least three, four, five, six, seven, eight, nine, ten, eleven, twelve, thirteen, fourteen, fifteen, sixteen, seventeen, eighteen, nineteen, twenty, 25, 30, 35, 40, 45, 50, 60, 70, 80, 90, 100, 120, 140, 160, 180, 200, 225, or 250 of the genes listed above. The person of ordinary skill in the art will understand the number of genes making up the plurality of genes may be different between different tumor types, but the number of genes can be readily selected as a matter of routine experimentation in light of the teachings of the present specification.

In one embodiment, the plurality of genes comprises those listed in Table 1:

TABLE 1

CTSA
ZNF175
SUMF2
MEPCE
TOM1
GPX4
ANO10
MFAP2
PGLYRP2
ETV2
THBS3
LPPR2
SNX21
ICAM3
FTO
FIBP
THYN1
PHYH
TK2
RNF187
F8
C14orf45
ABI3BP
RSPO3
MAN1B1
PHPT1
NEIL2
UBXN6
ATRIP
SNAPC2
NR1D1
TRPC3
hCG 20036
HOXB7
C7org106
AGXT2L2
SP4
ZNF280C
ZMYM2
HOXA5
LYN
CPVL
HOXA13
WT1
HOXA10
ZNF195
GPX7
CEP152
KCNK1
MOSC2
HOXA3
C15orf5
FLJ10213
C1orf115
ANKRD6
RGS10
C1orf31

The plurality of genes listed in Table 1 may be particularly useful when the patient's tumor is a central nervous system (CNS) tumor.

In one embodiment, the plurality of genes comprises those listed in Table 2:

TABLE 2

PCDHB11
PCDHB8
C4orf32
ATF7IP
PCDHB2
PUS3
RASSF8
DNM1L
CBX5
TRPS1
PCDH18
IGFBP3
NDRG1
NFIC
AKR1C3
MVP
HK1
FSCN1
CD44
RBMS3
DEPDC7
MYADM
ZEB1
SERPINH1
PTRF
NNMT
TNFRSF1A
WNT5A
MECOM
PFTK1
ANTXR2
ZNF462
PPAP2C
HMGA2
SPAG4
CPT1C
SERGEF
KIAA1324L
GARNL4
PCDH96
OTUB1
ZNF561
PLAG1
TBRG1
HOXB9
HOXA6
UBASH3B
BCL9L
ITPKB
GPR137B
CD83
SORBS1
SOX8
C17orf76
C21orf91
HIST1H2BH
CCDC30
NME5
FSTL5
TMEM60
CYP4V2
BR13
TMEM170B
CA14
C2orf88
RGS20
ITGA7
SLC16A6
DCDC2
SLC27A3
GPR143
IL12RB2
TTYH2
SEMA6A
RAB17
FAM174B
RAB11FIP4
TM4SF18
PAEP
HPS4
HHAT
SEPT3
ATP6V0D2
C10orf11
SLC44A3
GMPR
MAP4K3
GALNT3
LOC202781
ATP6V0A2
MICAL3
MAGEH1
SH3YL1
NINL
TSC22D3
FAM178A
ZBTB41
SLC17A5

The plurality of genes of Table 2 may be particularly useful when the patient's tumor is a melanoma.

In one embodiment, the plurality of genes comprises those listed in Table 3, below.

TABLE 3

ANKRD5
F2R
FSCN1
AKR1C3
AKR1C1
OSCP1
GNA12
ITGA5
BAD
RRAS
NRP1
WNT5A
ZFP36L1
EHD2
ANXA1
TNKS1BP1
CCDC109B
ZBTB47
NFASC
ST6GALNAC
KCTD17
FKBP10
KIAA0652
ARC
PCDHB16
PTMS
ARL2
THYN1
ROM1
SH3BGRL3
RAB1B
PRRX1
MAPKAPK3
PITPNM1
POLD4
KCNN4
MED16
PPP2R1A
PNPLA6
CALCOCO2
RTN4
MYOM2
MBL1P1
SMOX
PDCD2
LRRC29
C20orf134
NUDT22
NDUFV1
KIF23
SKA2
ANLN
TOB1
INTS2
RC3H1
PUM2
NINL
RAB15
SLC2A3P1
PGBD5
AUTS2
PCF11
SFRS15
KIAA0355
TUG1
POGK
ZXDB
PHLPP2
C6orf226
MOSPD1
RCOR3
NCOA6
ZNF138
NEO1
DDX60L
CCDC45
SPAG9
LRRC39
DUS1L
PDE8A
LINS1
BLM
LOC401068
NCALD
LOC152217
MRS2
KIAA1598
MGAT4A
C7orf31
CHD1
DAPK1
PPM1H
CHKA
SYCP2
IVNS1ABP
MESDC1
CDK5R1
DHX33

The plurality of genes of Table 3 may be particularly useful when the patient's tumor is of a variety of types, including CNS tumors and melanomas.

In one embodiment, the plurality of genes comprises those listed in Table 4, below.

TABLE 4

ROM1
PRRX1
TNKS1BP1
ZFP36L1
RRAS
NRP1
WNT5A
BAD
ANXA1
CCDC109B
ST6GALNAC
AKR1C3
FSCN1
PITPNM1
RTN4
THYN1
ARL2
KCTD17
FKBP10
ZXDB
C6orf226
MOSPD1
INTS2
ZNF138
CCDC45
NINL
KIAA0355
C7orf31
NEO1
RAB15
MYOM2
LINS1
DAPK1
PPM1H
NCALD
KIAA1598
MGAT4A
CHD1
IVNS1ABP
DHX33

The plurality of genes of Table 4 may be particularly useful when the patient's tumor is of a variety of types, including CNS tumors and melanomas.

In one embodiment, the plurality of genes comprises those listed in Table 5, below.

TABLE 5

MAN1B1
THYN1
FIBP
ZNF175
ETV2
UBXN6
MFAP2
NR1D1
THBS3
RNF187
TOM1
MEPCE
CTSA
PHYH
LPPR2
TK2
TK1
SNAPC2
ICAM3
C14orf45
GPX4
F8
ANO10
FTO
HOXA10
WT1
SP4
GPX7
TRPC3
HOXA3
C15orf5
FLJ10213
LYN
C1orf115
CPVL
ANKRD6
RGS10
ZMYM2
PLCL2
C14orf106
MOSC2
HOXA5
KCNK1
HOXB7
ZNF195

The plurality of genes of Table 5 may be particularly useful when the patient's tumor is a CNS tumor.

In one embodiment, the plurality of genes comprises those listed in Table 6, below.

TABLE 6

PAEP
TSC22D3
HPS4
HHAT
GPR143
ATP6V0A2
NINL
SLC17A5
IL12RB2
NME5
FAM174B
CA14
HIST1H2BH
ITM2C
SEMA6A
SLC27A3
SH3YL1
CD83
MAGEH1
RGS20
GMPR
RAB17
ITGA7
ITPKB
SLC16A6
SORBS1
MICAL3
MAP4K3
FAM178A
GALNT3
GPR137B
C21orf91
SERGEF
DNM1L
ATF7IP
CD44
PETK1
OTUB1
HK1
FSCN1
GARNL4
ZEB1
PTRF
HOXB9
PLAG1
RASSF8
HMGA2
MECOM
PUS3
PPAP2C
AKR1C3
WNT5A
MVP
TNFRSF1A
NFIC
RBMS3
CBX6
PCDHB6
PCDHB11
HOOK1
HOXA6
PCDHB8
KCNA4
SERPINH1
NNMT
IGFBP3
NDRG1
SPAG4
TRPS1

The plurality of genes of Table 6 may be particularly useful when the patient's tumor is a melanoma.

In one embodiment, the plurality of genes comprises those listed in Table 7, below.

TABLE 7

SFRS15
AUTS2
MOSPD1
CHD1
TOB1
DUS1L
SLC2A3P1
PHLPP2
KIF23
POGK
PDE8A
MRS2
KIAA1598
ZXDB
SPAG9
NINL
PPM1H
NCALD
NEO1
PGBD5
NCOA6
CDK5R1
KIAA0355
SYCP2
TUG1
IVNS1ABP
PUM2
PCF11
CHKA
MGAT4A
DAPK1
RAB15
RCOR3
MBL1P1
NELL1
NFASC
ROM1
AKR1C1
PNLPA6
FKBP10
SH3BGRL3
NOL3
PTMS
PRRX1
KCTD17
PPP2R1A
THYN1
GNAI2
AKR1C3
ANXA1
ARC
ZFP36L1
EHD2
F2R
ITGA5
NRP1
RRAS
MYOM2
ARL2
CALCOCO2
MED16
KCNN4
CCDC109B
KIAA0652
MAPKAPK3
POLDIP2
ANKRD5
SMOX
FSCN1
POLD4
RAB1B
NDUFV1
RTN4
PDCD2
BAD
WNT5A

The plurality of genes of Table 7 may be particularly useful when the patient's tumor is of a variety of types, including CNS tumors and melanomas.

In one embodiment, the plurality of genes comprises those listed in Table 8, below.

TABLE 8

ZXDB
INTS2
ZNF138
CCDC45
KIAA1598
MGAT4A
RAB15
C7orf31
MOSPD1
NEO1
DAPK1
NINL
KIAA0355
DHX33
IVNS1ABP
PPM1H
NCALD
CHD1
LINS1
C6orf226
MYOM2
AKR1C3
THYN1
ZFP36L1
TNKS1BP1
ROM1
KCTD17
ANXA1
ST6GALNAC
FKBP10
CCDC109B
BAD
PITPNM1
FSCN1
RRAS
WNT5A
NRP1
RTN4
PRRX1
ARL2

The plurality of genes of Table 8 may be particularly useful when the patient's tumor is a melanoma.

In one embodiment, the plurality of genes comprises those listed in Table 9, below.

TABLE 9

DAPK1
PDE8A
NINL
SYCP2
NCALD
CDK5R1
IVNS1ABP
BLM
C6orf226
LINS1
DDX60L
CHD1
PUM2
SLC2A3P1
INTS2
CHKA
SPAG9
MRS2
LOC152217
RAB15
DUS1L
MGAT4A
TOB1
KIAA1598
PCF11
POGK
RCOR3
ZXDB
NEO1
PHLPP2
KIAA0355
KIF23
LRRC39
DHX33
PPM1H
MESDC1
AUTS2
PGBD5
SKA2
ZNF138
CCDC45
SFRS15
NCOA6
LOC401068
C7orf31
ANLN
MOSPD1
RC3H1
TUG1
FAM129B
SMOX
RAB1B
POLD4
C20orf134
AKR1C3
AKR1C1
ANKRD5
MYOM2
NUDT22
NDUFV1
ZFP36L1
CALCOCO2
PITPNM1
MAPKAPK3
THYN1
KIAA0652
FSCN1
BAD
ARL2
OSCP1
CCDC109B
NFASC
MED16
PRRX1
KCNN4
RTN4
PCDHB16
RRAS
SH3BGRL3
WNT5A
NRP1
ITGA5
PNPLA6
LRRC29
PTMS
EHD2
FKBP10
ANXA1
GNAI2
F2R
ST6GALNAC
PPP2R1A
ZBTB47
ARC
TNKS1BP1
MBL1P1
ROM1
KCTD17

The plurality of genes of Table 9 may be particularly useful when the patient's tumor is of a variety of types, including CNS tumors and melanomas.

In one embodiment, the plurality of genes comprises those listed in Table 10, below.

TABLE 10

MAGEH1
RGS20
SLC16A6
CD83
HHAT
CCDC30
PAEP
ATP6V0D2
FSTL5
TM4SF18
MICAL3
ZBTB41
SLC17A5
HIST1H2BH
BRI3
TMEM60
ATP6VOA2
SOX8
SORBS1
SEMA6A
SLC27A3
ITGA7
CA14
CYP4V2
ITPKB
HPS4
GMPR
GPR137B
C21orf91
RAB17
MAP4K3
GPR143
TTYH2
C10orf11
IL12RB2
NME5
FAM178A
TMEM170B
NINL
C20orf88
SEPT3
C17orf76
DCDC2
GALNT3
FAM174B
TSC22D3
LOC202781
SH3YL1
RAB11FIP4
SLC44A3
MYADM
C4orf32
SPAG4
KIAA1324l
HK1
ZEB1
NNMT
CPT1C
SERGEF
RASSF8
ZNF462
NFIC
PCDHB2
PCDHB11
PCDHB8
PCDH18
AKR1C3
MVP
HOXA6
PLAG1
BCL9L
MECOM
HOXB9K
PPAP2C
OTUB1
DNM1L
GARNL4
SERPINH1
ANTXR2
IFGBP3
NDRG1
WNT5A
RBMS3
UBASH3B
HMGA2
CD44
TNFRSF1A
TBRG1
ATF7IP
CBX5
ZNF561
DEPDC7
PUS3
FSCN1
PFTK1
PTRF
TRPS1
PCDHB6

The plurality of genes of Table 10 may be particularly useful when the patient's tumor is a melanoma.

In one embodiment, the plurality of genes comprises those listed in Table 11, below.

TABLE 11

PHYH
GPX4
SUMF2
FTO
THYN1
ATRIP
MEPCE
RNF187
NEIL2
PHPT1
MAN1B1
ICAM3
ABI3BP
ZNF175
TK2
F8
LPPR2
ANO10
SNX21
C14orf45
TOM1
CTSA
MFAP2
NR1D1
THBS3
UBXN6
RSPO3
SNAPC2
ETV2
PGLYRP2
C14orf106
HOXA5
ZNF280C
C1orf31
ZNF195
C15orf5
HOXA3
CEP152
C7orf31
HOXA13
TRPC3
SP4
FLJ10213
CPVL
RGS10
ANKRD6
PLCL2
MOSC2
C1orf115
LYN
HOXB7
GPX7
AXGT2L2
HOXA10
ZMYM2
KCNK1
hCG 20036
WT1

The plurality of genes of Table 11 may be particularly useful when the patient's tumor is of a variety of types, including CNS tumors and melanomas.

In one embodiment, the plurality of genes comprises those listed in Table 12, below.

TABLE 12

PPM1H
MGAT4A
RAB15
MOSPD1
C6orf226
C7orf31
KIAA1598
KIAA0355
NEO1
NCALD
LINS1
ZNF138
CHD1
DAPK1
ZXDB
NINL
DHX33
IVNS1ABP
CCDC45
INTS2
PITPNM1
TNKS1BP1
MYOM2
PRRX1
ROM1
FKBP10
ST6GALNAC
KCTD17
AKR1C3
NRP1
BAD
RTN4
CCDC109B
RRAS
ZFP36L1
WNT5A
ANXA1
FSCN1
THYN1
ARL2

The plurality of genes of Table 12 may be particularly useful when the patient's tumor is of a variety of types, including CNS tumors and melanomas.

In one embodiment, the plurality of genes comprises those listed in Table 13, below.

TABLE 13

KIAA0355
KIF23
TOB1
SKA2
RC3H1
ZNF138
CHD1
PCF11
DAPK1
ZXDB
NCOA6
PUM2
MRS2
DHX33
LOC152217
SPAG9
CCDC45
INTS2
IVNS1ABP
RCOR3
AUTS2
LINS1
DUS1L
MESDC1
SLC2A3P1
TUG1
BLM
CHKA
SFRS15
POGK
CDK5R1
NINL
NEO1
SYCP2
LRRC39
NCALD
PGBD5
PPM1H
MGAT4A
RAB15
C7orf31
DDX60L
KIAA1598
PDE8A
C6orf226
LOC401068
PHLPP2
MOSPD1
ANLN
PDCD2
PTMS
FSCN1
ARL2
PPP2R1A
PITPNM1
KIAA0652
THYN1
NDUFV1
MED16
TNKS1BP1
MYOM2
ARC
FKBP10
ROM1
C20orf134
NUDT22
SB3BGRL3
PNPLA6
BAD
CCDC109B
RTN4
CALCOCO2
MAPKAPK3
F2R
OSCP1
PCDHB16
ZBTB47
MBL1P1
NFASC
AKR1C1
SMOX
KCTD17
ANKRD5
PRRX1
WNT5A
ANXA1
ZFP36L1
FAM129B
KCNN4
POLD4
RRAS
ITGA5
EHD2
NRP1
RABG1G
AKR1C3
GNAI2
ST6GALNAC
LRRC29

The plurality of genes of Table 13 may be particularly useful when the patient's tumor is of a variety of types, including CNS tumors and melanomas.

In one embodiment, the plurality of genes comprises those listed in Table 14, below.

TABLE 14

FIBP
GPX4
ATRIP
PHPT1
MAN1B1
PGLYRP2
MEPCE
RNF187
MFAP2
THYN1
NEIL2
FTO
ZNF175
THBS3
RSPO3
ABI3BP
ANO10
F8
PHYH
CTSA
TIK2
TOM1
ICAM3
SNX21
C14orf45
ETV2
SNAPC2
LPPR2
SUMF2
NR1D1
UBXN6
FLJ10213
AGXT2L2
C1orf115
hCG 20036
KCNK1
ZNF195
CEP152
ZNF280C
ZMYM2
C14orf106
WT1
C1orf31
RGS10
C7orf31
LVN
CPVL
SP4
GPX7
HOXA10
HOXA13
TRPC3
HOXB7
HOXA5
HOXA3
C15orf5
PLCL2
MOSC2
ANKRD6

The plurality of genes of Table 14 may be particularly useful when the patient's tumor is a CNS tumor.

In one embodiment, the plurality of genes comprises those listed in Table 15, below.

TABLE 15

HHAT
NME5
SLC17A5
GALNT3
RGS20
BRI3
CYP4V2
TMEM60
FAM178A
MICAL3
TSC22D3
CD83
SEMA6A
GPR137B
HIST1H2BH
IL12RB2
SORBS1
CA14
GPR143
C2orf88
ITGA7
C17orf76
C10orf11
ATP6V0D2
SLC44A3
TMEM170B
RAB11FIP4
SEPT3
ATP6V0A2
ZBTB41
MAGEH1
LOC202781
MAP4K3
SLC16A6
FSTL5
SOX8
FAM174B
TM4SF18
CCDC30
RAB17
PAEP
SLC27A3
GMPR
ITPKB
NINL
TTYH2
C21orf91
SH3YL1
HPS4
DCDC2
AKR1C3
MYADM
HK1
SPAG4
NDRG1
NNMT
MVP
IGFBP3
TNFRSF1A
WNT5A
NFIC
BCL9L
CD44
PTRF
PFTK1
PPAP2C
MECOM
UBASH3B
HOXB9
PCDHB8
PDCHB11
TBRG1
PCDHB2
RBMS3
PCDHB6
CPT1C
FSCN1
ZNF462
CBX5
DNM1L
OTUB1
ZEB1
GARNL4
TRPS1
PLAG1
ATF7IP
PCDH18
SERPINH1
ANTXR2
IKAA1324L
C4orf32
DEPDC7
SERGEF
HMGA2
RASSF8
HOXA6
PUS3
ZNF561

The plurality of genes of Table 15 may be particularly useful when the patient's tumor is of a variety of types, including CNS tumors and melanomas.

An “expression level” of a gene is the amount of a transcription or a translation product of the gene in a particular cell (or population of substantially identical cells) at a particular point in time. In general, methods of gene expression profiling include two large groups: methods based on hybridization analysis of polynucleotides, and methods based on sequencing of polynucleotides. The most commonly used methods known in the art for the quantification of mRNA expression in a sample include northern blotting and in situ hybridization; RNAse protection assays; and reverse transcription polymerase chain reaction (RT-PCR). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

Amounts of a translation product of a gene can be quantified, either in absolute or relative terms, by ELISA or ELISA-like assays; mass spectrometry-based techniques, such as isotope-coded affinity tags, isobaric labeling, metal coded tags, N-terminal labelling, stable isotope labeling with amino acids in cell culture (SILAC), label-free quantification; or two-dimensional gel electrophoresis, among others.

Two biological processes commonly involved in tumorigenesis include gene amplification and DNA methylation. Both processes result in the abnormal expression of genes important in tumor formation or progression. Techniques that monitor gene amplification and DNA methylation can therefore be considered techniques for determining an expression level of a gene expression.

In the present invention, expression levels may be determined by any techniques known to the person of ordinary skill in the art, such as microarrays, quantitative RT-PCR, Northern blots, and Western blots, among others, such as ELISA-like assays, quantum-dot gene expression analysis, or direct digital detection for gene expression analysis (NanoString Technologies, Seattle, Wash.). An expression level of a gene may be defined on absolute terms, such as by the amount of signal generated by a microarray, PCR process, or blot, or on relative terms, such as by normalizing an amount of signal to that of a control gene or genes, the same gene in a non-tumor cell, or to other standards. The expression level of the same gene in a tumor cell known to be sensitive to or resistant to an oncolytic virus treatment can be termed a “reference” expression level.

Microarray gene expression analysis can be particularly useful, in that it allows the simultaneous analysis of, potentially, thousands of genes. Also of particular use can be quantitative RT-PCR methods, which have been reported to be highly sensitive, precise, and reproducible.

Differential gene expression can also be identified, or confirmed using the microarray technique. Thus, the expression profile of various genes can be measured in either fresh or paraffin-embedded tumor tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines, and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels. Microarray analysis can be performed by commercially available equipment, following manufacturer's protocols, such as by using the Affymetrix GenChip technology, or Incyte's microarray technology.

The development of microarray methods for large-scale analysis of gene expression makes it possible to search systematically for molecular markers of cancer classification and outcome prediction in a variety of tumor types.

Of the techniques listed above, the most sensitive and most flexible quantitative method is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. The starting material is typically total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, etc., tumor, or tumor cell lines, with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A (1987), and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Foster City, Calif.), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif.), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is detected at the CCD. The system includes software for running the instrument and for analyzing the data.

5′-Nuclease assay data are initially expressed as C_{T}, or the threshold cycle. As discussed above, fluorescence values are recorded during every cycle and represent the amount of product amplified to that point in the amplification reaction. The point when the fluorescent signal is first recorded as statistically significant is the threshold cycle (C_{T}).

To minimize errors and the effect of sample-to-sample variation, RT-PCR is usually performed using one or more reference genes as internal standards. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPD) and β-actin (ACTB).

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

The steps of a representative protocol for profiling gene expression using fixed, paraffin-embedded tissues as the RNA source, including mRNA isolation, purification, primer extension and amplification are known in the art. Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR.

Reagents for measuring an expression level for each of a plurality of genes include those set forth above and others that will be apparent to the person of ordinary skill in the art having the benefit of the present disclosure. For example, reagents required for RT-PCR of a particular gene include a gene-specific reverse transcriptase promoter and gene-specific amplification primers. The person of ordinary skill in the art can readily identify such gene-specific reagents in view of the gene of interest and its sequence, which may be publicly available or can be routinely identified. For another example, reagents required for a microarray include gene-specific probes for each of the genes for which determination of expression levels is desired.

Once reference expression levels for each of the plurality of genes in both the first plurality of tumor lines or tumor tissues and the second plurality of tumor lines or tumor tissues are known, a threshold value for each gene can be set equal to a measure of central tendency between the expression levels in the first plurality of tumor lines or tumor tissues and the second plurality of tumor lines or tumor tissues. “Measure of central tendency” is used herein to refer to any value calculable from a set of data that is indicative of the central tendency of the set of data. A median and a mean are exemplary measures of central tendency, but others may be used, such as the nth percentile (with exemplary values of n being from 25 to 75, such as from 33 to 67, 40 to 60, 45 to 55, or about 50), among others. As the person of ordinary skill in the art is aware, medians and nth percentiles are relatively less sensitive to outliers than are means.

In addition, the measure of central tendency may be further processed, for example, by normalizing, such as by subtracting the average expression of a group of control genes not included in the plurality of genes from the raw value of the measure of central tendency.

As a hypothetical example of setting the threshold value for the gene, consider a first set of three tumor lines or tumor tissues, wherein the expression levels of the gene (in arbitrary units) are 120, 140, and 150, and a second set of three tumor lines or tumor tissues, wherein the expression levels of the gene are 20, 40, and 50, with the further condition that the average expression of a panel of control genes across all six tumor lines or tumor tissues is defined as 100. The median expression level of the gene in the first set of tumor lines or tumor tissues is 140 and the median expression level of the gene in the second set of tumor lines or tumor tissues is 40. The mean of the two expression levels is 90, which can be set as the threshold. If desired, the mean of the two expression levels can be further normalized by subtracting the average expression of the group of control genes, to yield a normalized mean of −10, which can be set as the threshold.

Also, a weight for each gene can be set equal to a measure of the variability of the expression levels within the first plurality of tumor lines or tumor tissues and within the second plurality of tumor lines or tumor tissues. The measure of variability may be a standard deviation, a root mean square successive difference, or the like. The weight can be inversely proportional to the measure of variability, i.e., the gene can be given more weight if the variability of its expression with the first plurality of tumor lines or tumor tissues and within the second plurality of tumor lines or tumor tissues is relatively low. A relatively low variability of expression can be considered an indicator of higher quality, less noisy expression level data. In one embodiment, the weight will be in the range of 0 to +∞.

Also, a multiplier for each gene can be set equal to 1, if a measure of central tendency of the expression levels of the gene in the first plurality of tumor lines or tumor tissues are greater than the threshold value, or equal to −1, if a measure of central tendency of the expression levels of the gene in the second plurality of tumor lines or tumor tissues are greater than the threshold value.

An expression level for each of the plurality of genes in the tumor can then be determined, such as by one of the techniques discussed above. The expression level may be normalized by any appropriate technique, such as those set forth above.

A single-gene predictor score for each of the plurality of genes can be calculated from: the multiplier for the gene times (the expression level in the tumor minus the threshold value for the gene) divided by the weight for the gene. For example, in one embodiment, if the expression level of a first gene in the tumor is higher than the threshold value for the gene and the first gene is more highly expressed in the first plurality of tumor lines or tumor tissues than the second, the single-gene predictor score will be positive (multiplier=1, expression level in the tumor minus the threshold value>0, weight>0). For another example, in one embodiment, if the expression level of a second gene in the tumor is lower than the threshold value for the gene and the second gene is more highly expressed in the second plurality of tumor lines or tumor tissues than the first, the single-gene predictor score will be positive (multiplier=−1, expression level in the tumor minus the threshold value<0, weight>0).

For another example, in one embodiment, if the expression level of a third gene in the tumor is higher than the threshold value for the gene and the third gene is more highly expressed in the second plurality of tumor lines or tumor tissues than the first, the single-gene predictor score will be negative (multiplier=−1, expression level in the tumor minus the threshold value>0, weight>0). For another example, in one embodiment, if the expression level of a fourth gene in the tumor is lower than the threshold value for the gene and the fourth gene is more highly expressed in the first plurality of tumor lines or tumor tissues than the second, the single-gene predictor score will be negative (multiplier=1, expression level in the tumor minus the threshold value<0, weight>0).

From the single-gene predictor scores for each of the plurality of genes, a predictor score can be calculated from the sum of the single-gene predictor scores for the plurality of genes.

From the predictor score can be predicted, if the predictor score is greater than a predictor score threshold, that the treatment would have efficacy, and if the predictor score is less than a predictor score threshold, that the treatment would lack efficacy. In one embodiment, the predictor score threshold is set to zero, and a positive predictor score would yield a prediction that the treatment would have efficacy, whereas a negative predictor score would yield a prediction that the treatment would lack efficacy.

The person of ordinary skill in the art can readily set a predictor score threshold as a matter of routine experimentation in light of the present disclosure.

“Prediction” does not necessarily mean that every tumor for which the predictor score is greater than the predictor score threshold would be completely eliminated by the treatment and every tumor for which the predictor score is less than the predictor score threshold would be completely resistant to the treatment. The present method, however, does provide a clinician with information about the efficacy of the treatment that may be useful in formulating a treatment regimen for a patient's particular tumor that includes or excludes the treatment in question or increases or decreases the doses, frequency of doses, etc. of the treatment.

Although the method above has been described with reference to determining expression levels in tumor samples and comparing the same to reference values in tumor lines or tumor tissues, it can more generally be used on any sample, tumorous or otherwise, with comparison to reference values from corresponding lines or tissues. Using the method in this way may also provide information to predict the efficacy of an oncolytic virus treatment of a tumor. Such a tumor may be of a type found in the tissue of reference, but need not be. Alternatively or additionally, using the method in this way may also provide information regarding effects on the tissue of reference of oncolytic virus administration to the patient. For example, the severity of side effects of an oncolytic virus administration may be predicted. For another example, the severity of an infection of the patient by the oncolytic virus may be predicted.

In another embodiment, the present invention relates to a method, comprising:

Identifying whether a central nervous system tumor is of a proneural subtype, a mesenchymal subtype, or a third subtype;

predicting an oncolytic virus treatment to have efficacy if the tumor is of the proneural subtype; and

predicting the oncolytic virus treatment to lack efficacy if the tumor is of the mesenchymal subtype.

The various subtypes of CNS tumors, and techniques to identify them, are known to the person of ordinary skill in the art. For example, proneural subtypes and mesenchymal subtypes of CNS tumors can be identified by a person of ordinary skill in the art of pathology. What was unknown before our work was that CNS tumors of the proneural subtype are generally sensitive to oncolytic virus treatments, and CNS tumors of the mesenchymal subtype are generally resistant to oncolytic virus treatments.

In another embodiment, the present invention relates to a method, comprising:

identifying whether a lung tumor is of a small-cell subtype, a non-small-cell subtype, or a third subtype;

predicting an oncolytic virus treatment to have efficacy if the tumor is of the small-cell subtype; and

predicting the oncolytic virus treatment to lack efficacy if the tumor is of the non-small-cell subtype.

The various subtypes of lung tumors, and techniques to identify them, are known to the person of ordinary skill in the art. For example, a trained pathologist can identify small-cell and non-small-cell lung tumors. What was unknown before our work was that lung tumors of the small-cell subtype are generally sensitive to oncolytic virus treatments, and lung tumors of the non-small-cell subtype are generally resistant to oncolytic virus treatments.

In another embodiment, the present invention relates to a method, comprising:

identifying whether a tumor is a metastasis of a primary melanoma; and

predicting an oncolytic virus treatment of the melanoma to have more efficacy than the same treatment for the primary melanoma.

Primary melanomas and metastases thereof, and techniques to identify them, are known to the person of ordinary skill in the art. What was unknown before our work was that metastases of melanomas are generally more sensitive to oncolytic virus treatments than the primary melanoma.

In yet another embodiment, the present invention relates to a kit for predicting an efficacy of an oncolytic virus treatment for a tumor, the kit comprising:

reagents for measuring an expression level for each of a plurality of genes in a tumor sample obtained from a patient. The plurality of genes and the tumor are described above.

In a further embodiment, the kit comprises instructions for performing a method as described above.

The steps of this method are described above. The instructions can be in any form. For one example, the instructions can be in the form of a printed document. For another example, the instructions can be in the form of a file of any format stored in a computer readable medium. The computer readable medium can be, for example, downloadable content, a CD, a DVD, a Blu-Ray disc, an SD card, a flash memory stick, etc. The computer can be any desktop or notebook computer, smartphone, handheld computer, or the like using any operating system. File formats readable and/or executable by a computer will depend on the operating system and, often, various libraries or other software functioning on the computer. Generally, the medium, the instructions, and the computer can be routinely selected by the person of ordinary skill in the art having the benefit of the present disclosure. In one further embodiment, the kit comprises a computer readable medium encoded with instructions that, when executed by a computer, perform the method.

In the present invention, the treatment comprises administration of at least one oncolytic virus. In one embodiment, the oncolytic virus is an RNA oncolytic virus, meaning an oncolytic virus having RNA as its genomic material. In a further embodiment, the RNA oncolytic virus is a paramyxovirus. In many still further embodiments, the paramyxovirus is a Newcastle Disease Virus (NDV). Exemplary treatments comprising administration of at least one oncolytic virus, such as NDV, are disclosed in U.S. patent application Ser. No. 12/776,488, filed May 10, 2010, assigned to the same assignee, and hereby incorporated herein by reference.

Generally speaking, an oncolytic virus, as used herein, is a virus that is able to infect and lyse cancer cells. Replication of an oncolytic virus can both facilitate tumor cell destruction and produce dose amplification at the tumor.

In one embodiment, the treatment may also comprise administering an immunostimulatory agent. In a further embodiment, the immunostimulatory agent is selected from the group consisting of (i) a CTLA-4 blocking agent that specifically binds to the extracellular domain of CTLA-4 and blocks the binding of CTLA-4 to CD80 or CD86; (ii) interleukin-21 (IL-21); (iii) anti-CD40; (iv) granulocyte-macrophage colony stimulating factor (GM-CSF); and two or more thereof.

Any mammal having a tumor may receive the oncolytic virus and the immunostimulatory agent, if any. The mammal may be man or a mammal having economic or esthetic utility for man, e.g., farm animals, service animals, or pets. In one embodiment, the mammal is selected from the group consisting of man, non-human primates, ovines, bovines, equines, porcines, canines, felines, mice, and rats.

In one embodiment, the tumor is in an organ selected from the group consisting of brain, lung, skin, mouth, esophagus, stomach, small intestine, large intestine, colon, liver, kidney, breast, ovary, prostate gland, testicle, pancreas, bladder, and lymph node.

The oncolytic virus and the immunostimulatory agent, if any, may be administered to the mammal via the same route or via different routes. In one embodiment, the oncolytic virus is administered into the tumor, for example, by intratumoral injection, and the immunostimulatory agent is administered systemically, for example, intravascularly, subcutaneously, peritoneally, etc.

Alternative routes for administering the oncolytic virus include intravenous injection, intramuscular injection, inhalation (which may be particularly suitable for tumors of the lung), or rectal (which may be particularly suitable for tumors of the large intestine or colon).

Dosage levels of the oncolytic virus may be routinely selected by a physician or veterinarian. Desirably, the dosage of the oncolytic virus is large enough to rapidly bring about a desired therapeutic response, but small enough to not be toxic to the patient. (“Toxic to the patient” here includes typical symptoms of drug- or radiation-based anticancer therapies, such as severe nausea and hair loss, as well as typical symptoms of NDV infection of birds, such as severe respiratory disease, severe digestive-tract lesions, neurological damage, and symptoms of overdose of NDV to humans, such as dyspnea, diarrhea, dehydration, transient thrombocytopenia, and diffuse vascular leak. Mild fever, conjunctivitis, and other transient flu-like symptoms are not “toxic to the patient”). For example, NDV strain PV-701 is well tolerated in patients with advanced solid cancers in doses of at least 3×10⁹ infectious particles by the i.v. route and of at least 4×10¹² by the intra-tumoral route. When patients were desensitized with a lower initial dose, the maximum tolerated dose (MTD) was increased about 10-fold.

In one embodiment, the oncolytic virus is administered by intratumoral injection once per week for about two to four months, followed by a maintenance regimen of intratumoral injection once about every two to four months.

The dosage of the immunostimulatory agent, if any is administered, may vary widely, depending upon the nature of the disease, the frequency of administration, the manner of administration, the purpose of the administration, the clearance of the agent from the host, and the like. The dosage administered will vary depending on known factors, such as the pharmacodynamic characteristics of the particular agent, mode and route of administration, age, health and weight of the recipient, nature and extent of symptoms, concurrent treatments, frequency of treatment and effect desired. The dose may be administered as infrequently as weekly or biweekly, or fractionated into smaller doses and administered daily, semi-weekly, etc. to maintain an effective dosage level. Generally, a daily dosage of active ingredient can be about 0.1 to 100 mg/kg of body weight. Dosage forms suitable for internal administration generally contain from about 0.1 mg to 500 mgs of active ingredient per unit. The active ingredient may vary from 0.5 to 95% by weight based on the total weight of the immunostimulatory agent.

Generally, the immunostimulatory agent, when administered according to a method of the present invention, can be efficacious in a lower dose than is typically observed for the immunostimulatory agent when administered without an oncolytic virus.

In one embodiment, the immunostimulatory agent is administered by intravenous injection in a dosage of about 10 mg/kg of body weight once about every two to four weeks for about two to four months, followed by a maintenance regimen of intravenous injection in a dosage of about 10 mg/kg of body weight once about every two to four months.

If both an oncolytic virus and an immunostimulatory agent are administered, the oncolytic virus may be administered to the mammal at a time before the immunostimulatory agent is administered. In one embodiment, the oncolytic virus is administered from 1 day to 5 days before the immunostimulatory agent is administered. Though not to be bound by theory, the oncolytic virus may play two roles. First, it may kill some tumor cells directly. Second, it may stimulate the immune system by leading to lysis of tumor cells it kills; tumor cell antigens released by lysis may then be picked up by dendritic cells and stimulate T cells, and thereby promote the killing of other tumor cells by the mammal's immune system. The immunostimulatory agent may promote the latter process. For example, a CTLA-4 blocking agent may reduce CTLA-4's activity of downregulating T cells. Viruses can also directly activate innate immunity by triggering toll-like receptors (TLRs) on immune cells.

In addition to administering the oncolytic virus and the immunostimulatory agent, in various embodiments, additional materials may be administered to the patient.

In one embodiment, to the mammal is administered an anticancer agent other than the oncolytic virus and the immunostimulatory agent. Any known anticancer agent can be administered by a route, in a dosage, and in a treatment regimen known to a person of ordinary skill in the art of anticancer therapy.

In one embodiment, the treatment further comprises administering to the mammal a radiation therapy. Any known radiation source can be administered by techniques, in a dosage, and in a treatment regimen known to a person of ordinary skill in the art of radiation therapy for cancer. The person of ordinary skill in the art may find, as a matter of routine experimentation, that a reduced dosage or shorter or less intensive treatment regimen of radiation therapy may be effective when performed as part of the present method than when radiation therapy is administered by itself.

The progress of treatment resulting from administration as described above can be routinely monitored by techniques known to the person of ordinary skill in the art, including, but not limited to, noninvasive imaging, biopsy, and analysis of blood-borne markers of tumor activity (generally correlated with tumor mass), among others.

In one example, the above techniques may be used to treat a first tumor, a second tumor, or both in a mammal having a first tumor. The example comprises administering an oncolytic virus into the first tumor, and administering an immunostimulatory agent systemically to the mammal.

A common cancer progression is for a primary tumor to arise in a particular tissue, organ, or organ system of a mammalian body. Subsequently, one or more metastases of the primary tumor may arise in other particular tissues, organs, or organ systems of the mammalian body, typically by migration of one or more cells of the primary tumor through the mammal's blood or lymph.

Frequently, it is more difficult to treat a metastatic cancer than a primary tumor. A metastatic cancer may occur at one or more locations unamenable to various treatment options, and/or in such a profusion of sites that various treatment options may be relatively ineffective. In addition, a metastatic cancer often arises when a primary tumor is relatively advanced and the patient's prognosis is already relatively poor.

Administering an oncolytic virus into a first tumor (which may be a primary tumor or a metastatic tumor) and administering an immunostimulatory agent systemically to a mammal can lead to a reduction in the size of the second tumor (which may be a metastatic tumor) greater than that found when administering an oncolytic virus into the first tumor alone or administering an immunostimulatory agent systemically to the mammal alone. To our knowledge, this result was first reported in U.S. patent application Ser. No. 12/776,488, filed May 10, 2010, assigned to the same assignee.

In one embodiment, the example further comprises administering a localized anticancer therapy to the first tumor. The localized anticancer therapy can be a radiation therapy, though alternatively or in addition, other localized anticancer therapies, such as targeted therapies, including targeted chemotherapy, can be used. Administering an oncolytic virus into a first tumor, administering an immunostimulatory agent systemically to a mammal, and administering a localized anticancer therapy to the first tumor can lead to a reduction in the size of the second tumor greater than that found when administering an oncolytic virus into the first tumor alone, administering an immunostimulatory agent systemically to the mammal alone, or administering a localized anticancer therapy to the first tumor alone.

Administering an oncolytic virus into a first tumor and administering an immunostimulatory agent systemically to a mammal can lead to a reduction in the size of the first tumor. Also, administering an oncolytic virus into a first tumor, administering an immunostimulatory agent systemically to a mammal, and administering a localized anticancer therapy to the first tumor can lead to a reduction in the size of the first tumor.

### Examples

NDV sensitivity for each of a set of melanoma tumor lines was assayed using known techniques. Reported results included IC₈₀ values for each line. Expression levels of a plurality of genes from the set of melanoma tumor lines were determined by known techniques. A melanoma-derived gene expression signature of NDV sensitivity was defined as the 50 genes most significantly induced in both in the three most NDV-sensitive lines relative to the three most NDV-resistant lines and in the four most NDV-sensitive lines relative to the four most NDV-resistant lines and the 50 genes most significantly repressed in the same comparison, yielding a signature of 100 genes total. Two comparisons were used to reduce the likelihood of noise over/understating the expression of a gene in one comparison.

Similarly, for a set of CNS tumor lines, NDV sensitivity and gene expression were determined. A CNS-derived gene expression signature of NDV sensitivity was defined from the intersection of two lists: a first list of 50 genes most significantly induced or repressed both in the three most NDV-sensitive lines relative to the three most NDV-resistant lines and in the four most NDV-sensitive lines relative to the four most NDV-resistant lines and a second list of 50 genes most significantly induced or repressed both in the four most NDV-sensitive lines relative to the four most NDV-resistant lines as quantified by IC₈₀, and in the four most NDV-sensitive lines relative to the four most NDV-resistant lines as quantified by IC₅₀. The intersection consisted of 31 genes expressed more highly in NDV-resistant lines and 29 genes expressed more highly in NDV-sensitive lines. Two comparisons were used to reduce the likelihood of noise over/understating the expression of a gene in one comparison.

A third, joint signature, consisting of the top 20 genes common to both melanoma and CNS tumors that were expressed more highly in NDV-resistant lines, and the top 20 genes common to both melanoma and CNS tumors that were expressed more highly in NDV-sensitive lines, was defined.

A fourth, joint signature, consisting of the top 50 genes common to both melanoma and CNS tumors that were expressed more highly in NDV-resistant lines, and the top 50 genes common to both melanoma and CNS tumors that were expressed more highly in NDV-sensitive lines, was also defined.

A total predictor score was calculated using the weighted-voting algorithm of Gollub to assign samples to an NDV sensitive group and an NDV resistant group. First, a training set of data was used to identify for each gene a threshold value that was the midpoint between the average expression values within the two groups; a weight that was a measure of the variability within each of the two groups; and whether expression values were higher than the threshold value in the NDV sensitive group or the NDV resistant group. To establish the final thresholds for use, the overall mean Then, for each gene in the sample, the amount by which the expression was greater or less than the threshold value was determined, divided by the weight, and multiplied by +1 if expression was higher in the NDV sensitive group or by −1 if expression was higher in the NDV resistant group. The resulting values were summed for all genes under consideration. A positive score predicted NDV sensitivity and a negative score predicted NDV resistance.

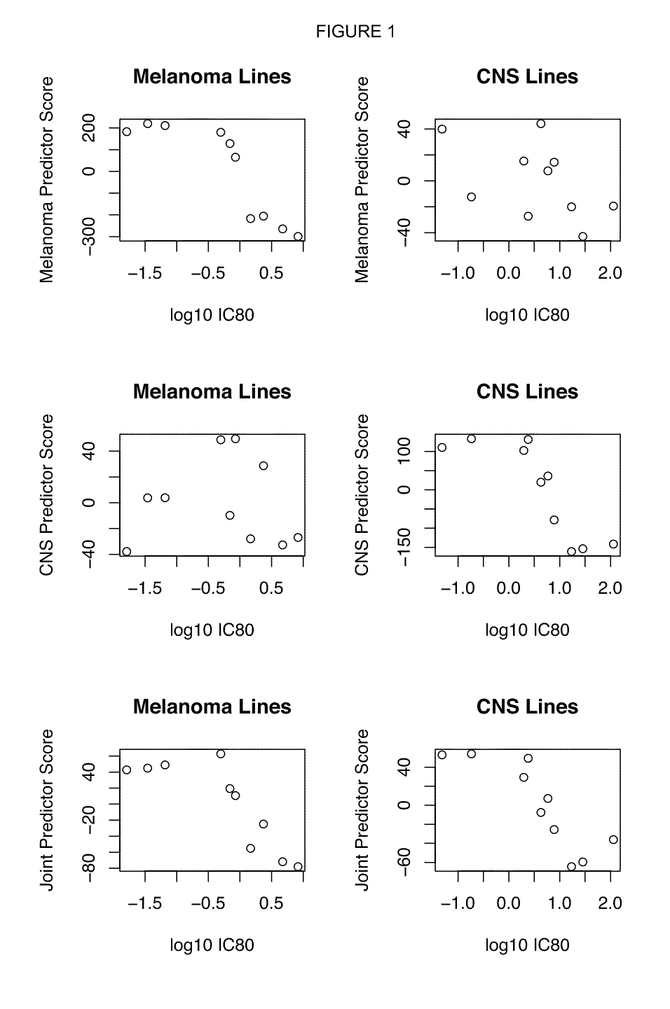

FIG. 1 shows predictor scores vs. log₁₀ of the NDV IC₈₀ for a plurality of melanoma and CNS lines using the melanoma signature, the CNS signature, and one of the joint signatures. Lower values of IC₈₀ indicate reduced amounts of the virus were more effective at inhibiting tumor line growth. As can be seen from FIG. 1, the plots for melanoma lines with the melanoma signature (upper left), CNS lines with the CNS signature (center right), melanoma lines with the joint signature (lower left), and CNS lines with the joint signature (lower right) were generally sigmoidal, with low IC₈₀ values correlated with predicted NDV sensitivity and high IC₈₀ values correlated with predicted NDV resistance.

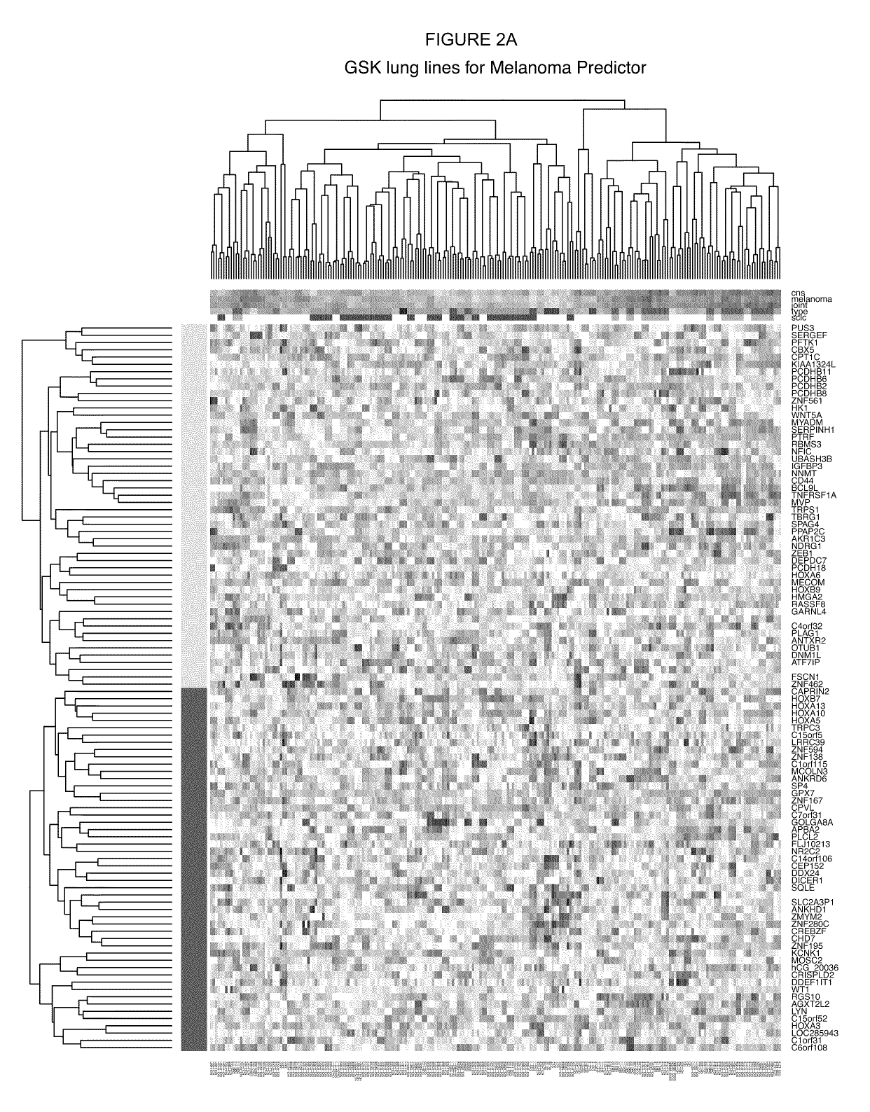

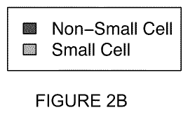

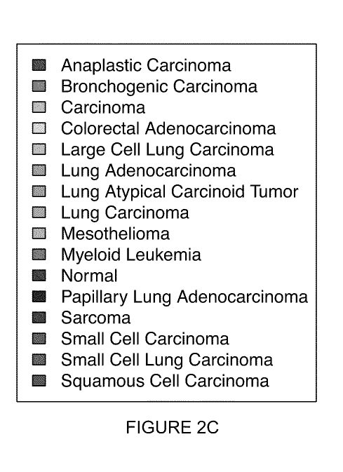

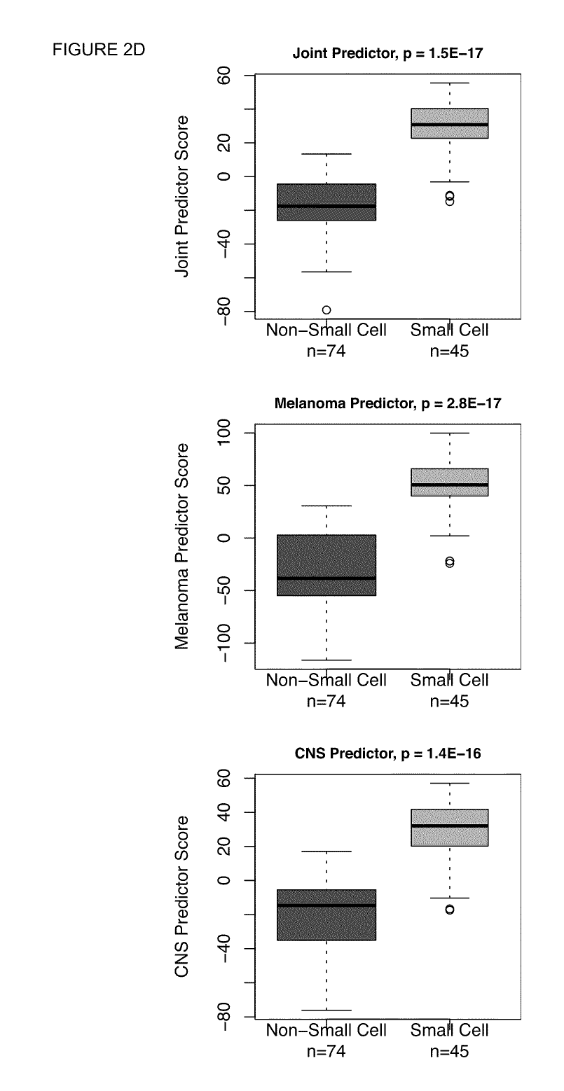

The three signatures were used to predict NDV sensitivity or resistance for lung tumor lines from the publicly available GSK data set. FIG. 2A shows a heatmap of increased expression (red) or decreased expression (blue) for various genes (rows) for various tumor lines (columns). The rows annotated in yellow are genes expressed more strongly in resistant lines and the rows annotated in purple are genes expressed more strongly in sensitive lines. The columns are annotated with the predicted sensitivity from each of the three signatures, with purple indicating sensitivity and yellow, resistance; tumor subtype (key shown in FIG. 2C); and small-cell (SCLC), non-small-cell (NSCLC), or neither (key shown in FIG. 2B).

FIG. 2D shows that all three signatures predicted SCLC to be far more NDV sensitive than NSCLC. The p-values were determined by a Wilcoxon Rank sum test.

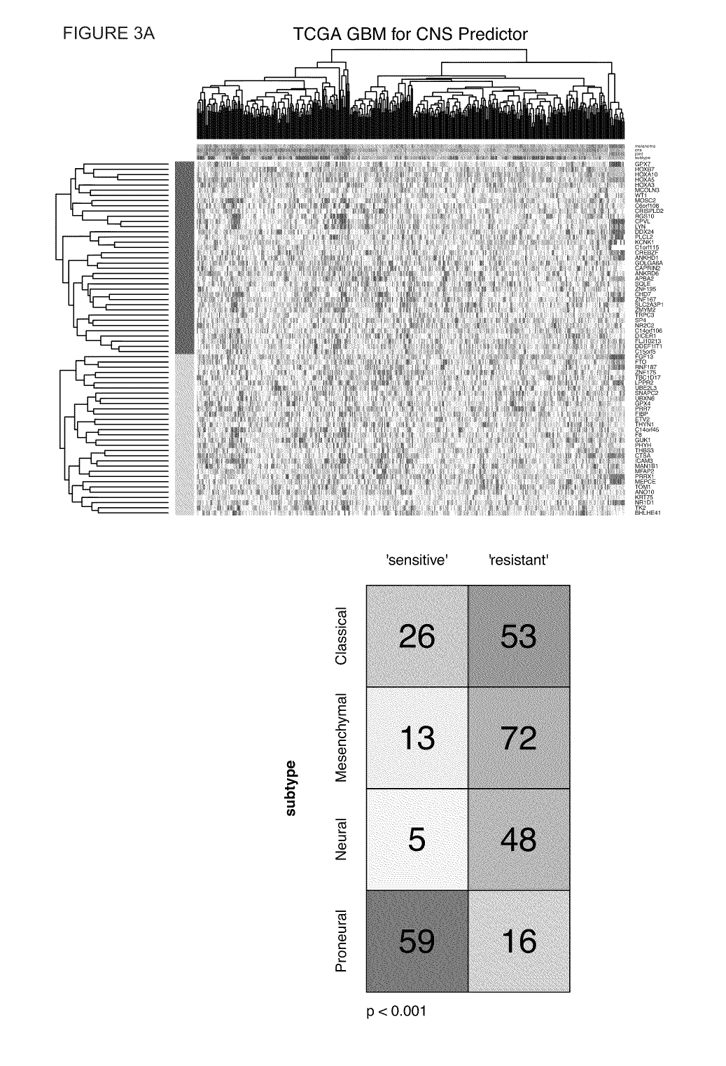

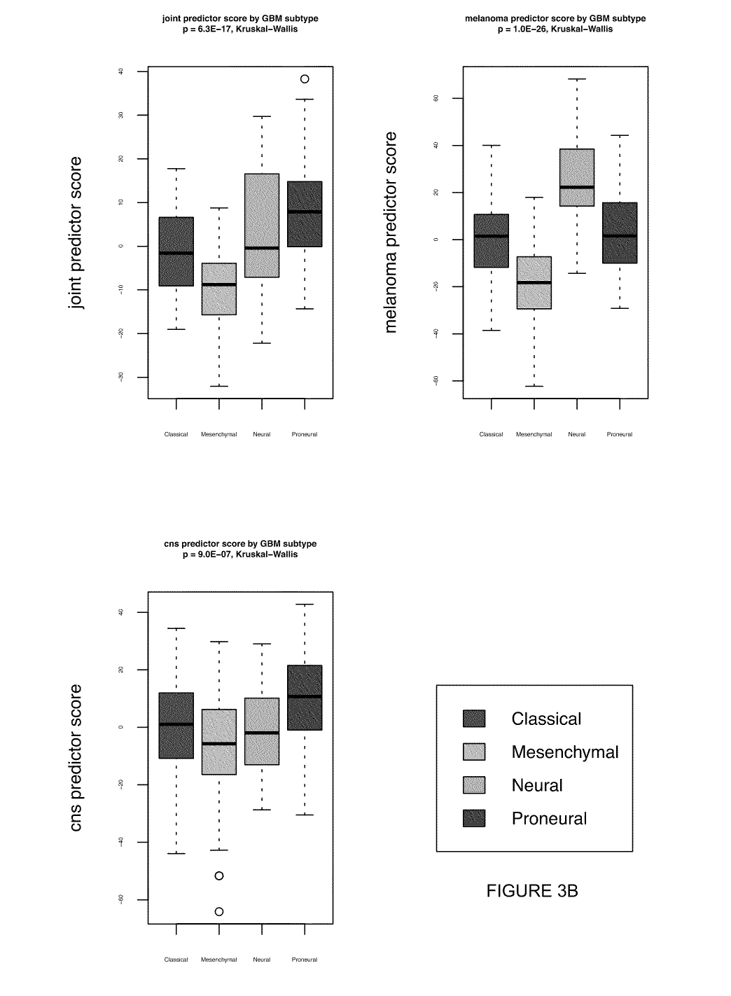

FIGS. 3A and 3B show similar use of the signatures to predict NDV sensitivity or resistance for glioblastoma tumor lines from the publicly available GSK data set. All three signatures predicted proneural tumors to be more NDV sensitive than mesenchymal tumors. The p-value for the table in FIG. 3A was determined by Fisher's Exact test; for FIG. 3B, the p-value was determined by a Wilcoxon Rank sum test.

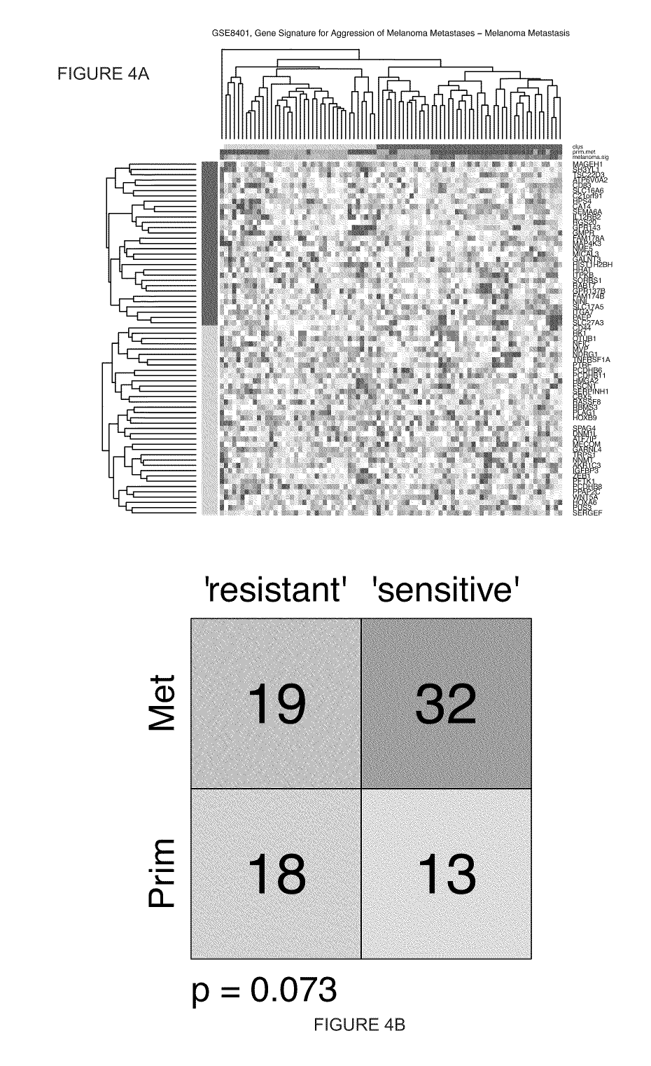

FIGS. 4A and 4B show similar use of the melanoma signature predict NDV sensitivity or resistance for primary melanoma lines and melanoma metastases. In FIG. 4A, the columns are annotated with a cluster identifier (purple for sensitive, yellow for resistant, based on hierarchical clustering), primary melanoma (cyan) or metastasis (red), and predicted NDV sensitivity. FIG. 4B shows metastases were more likely to be predicted to be sensitive than primary tumors.

All of the compositions and methods disclosed and claimed herein can be made and executed without undue experimentation in light of the present disclosure. While the compositions and methods of this invention have been described in terms of particular embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are chemically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method, comprising:
determining an expression level for each of a plurality of genes in a tumor sample obtained from a patient;
calculating a total predictor score based on the expression levels for each of the plurality of genes; and
predicting, if the total predictor score satisfies a first predictor score threshold, that an oncolytic virus treatment for the tumor would have efficacy, and if the total predictor score satisfies a second predictor score threshold, that the oncolytic virus treatment for the tumor would lack efficacy.

2. The method of claim 1, wherein the total predictor score is calculated from a single-gene predictor score for each of the plurality of genes, wherein each single-gene predictor score is calculated from a multiplier for the gene times (the expression level in the tumor sample minus a threshold value for the gene) divided by a weight for the gene,
wherein:
the threshold value for each gene is equal to a measure of central tendency between reference expression levels of the gene in a first plurality of tumor lines or tumor tissues known to be sensitive to the oncolytic virus treatment and a second plurality of tumor lines or tumor tissues known to be resistant to the oncolytic virus treatment;
the weight for each gene is equal to a measure of the variability of reference expression levels within the first plurality of tumor lines or tumor tissues and within the second plurality of tumor lines or tumor tissues; and
the multiplier for each gene is equal to 1, if a measure of central tendency of the reference expression levels of the gene in the first plurality of tumor lines or tumor tissues is greater than the threshold value, or −1, if a measure of central tendency of the reference expression levels of the gene in the second plurality of tumor lines or tumor tissues is greater than the threshold value.

3. The method of claim 1, wherein the oncolytic virus is an RNA oncolytic virus.

4. The method of claim 3, wherein the RNA oncolytic virus is a paramyxovirus.

5. The method of claim 4, wherein the paramyxovirus is a Newcastle Disease Virus (NDV).

6. The method of claim 1, wherein each of the first plurality of tumor lines or tumor tissues and the second plurality of tumor lines or tumor tissues are of the same tumor type as the tumor.

7. The method of claim 1, wherein the tumor is a tumor of the central nervous system, skin, bladder, breast, esophagus, kidney, liver, lung, ovary, pancreas, stomach, uterus, cervix, testicle, or colon, or a metastasis thereof.

8. The method of claim 1, wherein the plurality of genes comprises at least two of:
ABI3BP, AGXT2L2, AKR1C1, ANKRD5, ANLN, ANO10, ANTXR2, ANXA1, ARC, ARL2, ATF7IP, ATP6V0A2, ATRIP, AUTS2, AXGT2L2, BAD, BCL9L, BLM, BR13, C10orf11, C14orf106, C14orf45, C15orf5, C17orf76, C1orf115, C1orf31, C20orf134, C20orf88, C21orf91, C2orf88, C4orf32, C6orf226, C7orf31, C7org106, CA14, CALCOCO2, CBX5, CBX6, CCDC109B, CCDC30, CCDC45, CD44, CD83, CDK5R1, CEP152, CHD1, CHKA, CPT1C, CPVL, CTSA, CYP4V2, DAPK1, DCDC2, DDX60L, DEPDC7, DHX33, DNM1L, DUS1L, EHD2, ETV2, F2R, F8, FAM129B, FAM174B, FAM178A, FIBP, FKBP10, FLJ10213, FSCN1, FSTL5, FTO, GALNT3, GARNL4, GMPR, GNA12, GPR137B, GPR143, GPX4, GPX7, hCG 20036, HHAT, HIST1H2BH, HK1, HMGA2, HOOK1, HOXA10, HOXA13, HOXA3, HOXA5, HOXA6, HOXB7, HOXB9, HOXB9K, HPS4, ICAM3, IGFBP3, IKAA1324L, IL12RB2, INTS2, ITGA5, ITGA7, ITM2C, ITPKB, IVNS1ABP, KCNA4, KCNK1, KCNN4, KCTD17, KIAA0355, KIAA0652, KIAA1324, KIAA1598, KIF23, LINS1, LOC152217, LOC202781, LOC401068, LPPR2, LRRC29, LVN, MAGEH1, MAN1B1, MAP4K3, MAPKAPK3, MBL1P1, MECOM, MED16, MEPCE, MESDC1, MFAP2, MGAT4A, MICAL3, MOSC2, MOSPD1, MRS2, MVP, MYADM, MYOM2, NCALD, NCOA6, NDRG1, NDUFV1, NEIL2, NELL1, NEO1, NFASC, NFIC, NINL, NME5, NNMT, NOL3, NR1D1, NRP1, NUDT22, OSCP1, OTUB1, PAEP, PCDH18, PCDH96, PCDHB11, PCDHB16, PCDHB2, PCDHB6, PCDHB8, PCF11, PDCD2, PDCHB11, PDE8A, PFTK1, PGBD5, PGLYRP2, PHLPP2, PHPT1, PHYH, PITPNM1, PLAG1, PLCL2, PNLPA6, POGK, POLD4, POLDIP2, PPAP2C, PPM1H, PPP2R1A, PRRX1, PTMS, PTRF, PUM2, PUS3, RAB11FIP4, RAB15, RAB17, RAB1B, RABG1G, RASSF8, RBMS3, RC3H1, RCOR3, RGS10, RGS20, RNF187, ROM1, RRAS, RSPO3, RTN4, SB3BGRL3, SEMA6A, SEPT3, SERGEF, SERPINH1, SFRS15, SH3BGRL3, SH3YL1, SKA2, SLC16A6, SLC17A5, SLC27A3, SLC2A3P1, SLC44A3, SMOX, SNAPC2, SNX21, SORBS1, SOX8, SP4, SPAG4, SPAG9, ST6GALNAC, SUMF2, SYCP2, TBRG1, THBS3, THYN1, TIK2, TK1, TK2, TM4SF18, TMEM170B, TMEM60, TNFRSF1A, TNKS1BP1, TOB1, TOM1, TRPC3, TRPS1, TSC22D3, TTYH2, TUG1, UBASH3B, UBXN6, WNT5A, WT1, ZBTB41, ZBTB47, ZEB1, ZFP36L1, ZMYM2, ZNF138, ZNF175, ZNF195, ZNF280C, ZNF462, ZNF561, or ZXDB.

9. The method of claim 8, wherein the plurality of genes comprises the genes listed in Table 1, the genes listed in Table 2, the genes listed in Table 3, the genes listed in Table 4, the genes listed in Table 5, the genes listed in Table 6, the genes listed in Table 7, the genes listed in Table 8, the genes listed in Table 9, the genes listed in Table 10, the genes listed in Table 11, the genes listed in Table 12, the genes listed in Table 13, the genes listed in Table 14, the genes listed in Table 15, or two or more thereof.

10. A kit for predicting an efficacy of an oncolytic virus treatment for a tumor, the kit comprising:
reagents for measuring an expression level for each of a plurality of genes in a tumor sample obtained from a patient, wherein the plurality of genes comprises at least two of:
ABI3BP, AGXT2L2, AKR1C1, ANKRD5, ANLN, ANO10, ANTXR2, ANXA1, ARC, ARL2, ATF7IP, ATP6V0A2, ATRIP, AUTS2, AXGT2L2, BAD, BCL9L, BLM, BR13, C10orf11, C14orf106, C14orf45, C15orf5, C17orf76, C1orf115, C1orf31, C20orf134, C20orf88, C21orf91, C2orf88, C4orf32, C6orf226, C7orf31, C7org106, CA14, CALCOCO2, CBX5, CBX6, CCDC109B, CCDC30, CCDC45, CD44, CD83, CDK5R1, CEP152, CHD1, CHKA, CPT1C, CPVL, CTSA, CYP4V2, DAPK1, DCDC2, DDX60L, DEPDC7, DHX33, DNM1L, DUS1L, EHD2, ETV2, F2R, F8, FAM129B, FAM174B, FAM178A, FIBP, FKBP10, FLJ10213, FSCN1, FSTL5, FTO, GALNT3, GARNL4, GMPR, GNA12, GPR137B, GPR143, GPX4, GPX7, hCG 20036, HHAT, HIST1H2BH, HK1, HMGA2, HOOK1, HOXA10, HOXA13, HOXA3, HOXA5, HOXA6, HOXB7, HOXB9, HOXB9K, HPS4, ICAM3, IGFBP3, IKAA1324L, IL12RB2, INTS2, ITGA5, ITGA7, ITM2C, ITPKB, IVNS1ABP, KCNA4, KCNK1, KCNN4, KCTD17, KIAA0355, KIAA0652, KIAA1324, KIAA1598, KIF23, LINS1, LOC152217, LOC202781, LOC401068, LPPR2, LRRC29, LVN, MAGEH1, MAN1B1, MAP4K3, MAPKAPK3, MBL1P1, MECOM, MED16, MEPCE, MESDC1, MFAP2, MGAT4A, MICAL3, MOSC2, MOSPD1, MRS2, MVP, MYADM, MYOM2, NCALD, NCOA6, NDRG1, NDUFV1, NEIL2, NELL1, NEO1, NFASC, NFIC, NINL, NME5, NNMT, NOL3, NR1D1, NRP1, NUDT22, OSCP1, OTUB1, PAEP, PCDH18, PCDH96, PCDHB11, PCDHB16, PCDHB2, PCDHB6, PCDHB8, PCF11, PDCD2, PDCHB11, PDE8A, PFTK1, PGBD5, PGLYRP2, PHLPP2, PHPT1, PHYH, PITPNM1, PLAG1, PLCL2, PNLPA6, POGK, POLD4, POLDIP2, PPAP2C, PPM1H, PPP2R1A, PRRX1, PTMS, PTRF, PUM2, PUS3, RAB11FIP4, RAB15, RAB17, RAB1B, RABG1G, RASSF8, RBMS3, RC3H1, RCOR3, RGS10, RGS20, RNF187, ROM1, RRAS, RSPO3, RTN4, SB3BGRL3, SEMA6A, SEPT3, SERGEF, SERPINH1, SFRS15, SH3BGRL3, SH3YL1, SKA2, SLC16A6, SLC17A5, SLC27A3, SLC2A3P1, SLC44A3, SMOX, SNAPC2, SNX21, SORBS1, SOX8, SP4, SPAG4, SPAG9, ST6GALNAC, SUMF2, SYCP2, TBRG1, THBS3, THYN1, TIK2, TK1, TK2, TM4SF18, TMEM170B, TMEM60, TNFRSF1A, TNKS1BP1, TOB1, TOM1, TRPC3, TRPS1, TSC22D3, TTYH2, TUG1, UBASH3B, UBXN6, WNT5A, WT1, ZBTB41, ZBTB47, ZEB1, ZFP36L1, ZMYM2, ZNF138, ZNF175, ZNF195, ZNF280C, ZNF462, ZNF561, or ZXDB.

11. The kit of claim 10, wherein the plurality of genes comprises the genes listed in Table 1, the genes listed in Table 2, the genes listed in Table 3, the genes listed in Table 4, the genes listed in Table 5, the genes listed in Table 6, the genes listed in Table 7, the genes listed in Table 8, the genes listed in Table 9, the genes listed in Table 10, the genes listed in Table 11, the genes listed in Table 12, the genes listed in Table 13, the genes listed in Table 14, the genes listed in Table 15, or two or more thereof.

12. The kit of claim 10, wherein the tumor is a tumor of the central nervous system, skin, bladder, breast, esophagus, kidney, liver, lung, ovary, pancreas, stomach, uterus, cervix, testicle, or colon, or a metastasis thereof.

13. The kit of claim 10, further comprising instructions for performing a method comprising:
determining an expression level for each of the plurality of genes;
calculating a total predictor score based on the expression levels for each of the plurality of genes; and
predicting, if the total predictor score satisfies a first predictor score threshold, that an oncolytic virus treatment for the tumor would have efficacy, and if the total predictor score satisfies a second predictor score threshold, that the oncolytic virus treatment for the tumor would lack efficacy.

14. A method, comprising:
determining an expression level for each of a plurality of genes in a tumor sample by at least one of northern blotting, in situ hybridization, RNAse protection assay, reverse transcription polymerase chain reaction (RT-PCR), quantitative RT-PCR, use of an antibody to DNA duplexes, use of an antibody to RNA duplexes, use of an antibody to DNA-RNA hybrid duplexes, use of an antibody to DNA-protein duplexes, Serial Analysis of Gene Expression (SAGE), gene expression analysis by massively parallel signature sequencing (MPSS), a microarray, southern blotting, western blotting, ELISA-like assays, quantum-dot gene expression analysis, or direct digital detection for gene expression analysis,
wherein the plurality of genes comprises at least two of:
ABI3BP, AGXT2L2, AKR1C1, ANKRD5, ANLN, ANO10, ANTXR2, ANXA1, ARC, ARL2, ATF7IP, ATP6V0A2, ATRIP, AUTS2, AXGT2L2, BAD, BCL9L, BLM, BR13, C10orf11, C14orf106, C14orf45, C15orf5, C17orf76, C1orf115, C1orf31, C20orf134, C20orf88, C21orf91, C2orf88, C4orf32, C6orf226, C7orf31, C7org106, CA14, CALCOCO2, CBX5, CBX6, CCDC109B, CCDC30, CCDC45, CD44, CD83, CDK5R1, CEP152, CHD1, CHKA, CPT1C, CPVL, CTSA, CYP4V2, DAPK1, DCDC2, DDX60L, DEPDC7, DHX33, DNM1L, DUS1L, EHD2, ETV2, F2R, F8, FAM129B, FAM174B, FAM178A, FIBP, FKBP10, FLJ10213, FSCN1, FSTL5, FTO, GALNT3, GARNL4, GMPR, GNA12, GPR137B, GPR143, GPX4, GPX7, hCG 20036, HHAT, HIST1H2BH, HK1, HMGA2, HOOK1, HOXA10, HOXA13, HOXA3, HOXA5, HOXA6, HOXB7, HOXB9, HOXB9K, HPS4, ICAM3, IGFBP3, IKAA1324L, IL12RB2, INTS2, ITGA5, ITGA7, ITM2C, ITPKB, IVNS1ABP, KCNA4, KCNK1, KCNN4, KCTD17, KIAA0355, KIAA0652, KIAA1324, KIAA1598, KIF23, LINS1, LOC152217, LOC202781, LOC401068, LPPR2, LRRC29, LVN, MAGEH1, MAN1B1, MAP4K3, MAPKAPK3, MBL1P1, MECOM, MED16, MEPCE, MESDC1, MFAP2, MGAT4A, MICAL3, MOSC2, MOSPD1, MRS2, MVP, MYADM, MYOM2, NCALD, NCOA6, NDRG1, NDUFV1, NEIL2, NELL1, NEO1, NFASC, NFIC, NINL, NME5, NNMT, NOL3, NR1D1, NRP1, NUDT22, OSCP1, OTUB1, PAEP, PCDH18, PCDH96, PCDHB11, PCDHB16, PCDHB2, PCDHB6, PCDHB8, PCF11, PDCD2, PDCHB11, PDE8A, PFTK1, PGBD5, PGLYRP2, PHLPP2, PHPT1, PHYH, PITPNM1, PLAG1, PLCL2, PNLPA6, POGK, POLD4, POLDIP2, PPAP2C, PPM1H, PPP2R1A, PRRX1, PTMS, PTRF, PUM2, PUS3, RAB11FIP4, RAB15, RAB17, RAB1B, RABG1G, RASSF8, RBMS3, RC3H1, RCOR3, RGS10, RGS20, RNF187, ROM1, RRAS, RSPO3, RTN4, SB3BGRL3, SEMA6A, SEPT3, SERGEF, SERPINH1, SFRS15, SH3BGRL3, SH3YL1, SKA2, SLC16A6, SLC17A5, SLC27A3, SLC2A3P1, SLC44A3, SMOX, SNAPC2, SNX21, SORBS1, SOX8, SP4, SPAG4, SPAG9, ST6GALNAC, SUMF2, SYCP2, TBRG1, THBS3, THYN1, TIK2, TK1, TK2, TM4SF18, TMEM170B, TMEM60, TNFRSF1A, TNKS1BP1, TOB1, TOM1, TRPC3, TRPS1, TSC22D3, TTYH2, TUG1, UBASH3B, UBXN6, WNT5A, WT1, ZBTB41, ZBTB47, ZEB1, ZFP36L1, ZMYM2, ZNF138, ZNF175, ZNF195, ZNF280C, ZNF462, ZNF561, or ZXDB.

15. The method of claim 14, wherein the plurality of genes comprises the genes listed in Table 1, the genes listed in Table 2, the genes listed in Table 3, the genes listed in Table 4, the genes listed in Table 5, the genes listed in Table 6, the genes listed in Table 7, the genes listed in Table 8, the genes listed in Table 9, the genes listed in Table 10, the genes listed in Table 11, the genes listed in Table 12, the genes listed in Table 13, the genes listed in Table 14, the genes listed in Table 15, or two or more thereof.

16. The method of claim 14, wherein the tumor is a tumor of the central nervous system, skin, bladder, breast, esophagus, kidney, liver, lung, ovary, pancreas, stomach, uterus, cervix, testicle, or colon, or a metastasis thereof.
